(19) United States
(12) Patent Application Publication
(10) Pub. No.: US 2019/0201031 A1
(43) Pub. Date: Jul. 4, 2019

(54) SCISSOR FORCEPS

(71) Applicant: Olympus Corporation, Tokyo (JP)

(72) Inventor: Takashi Nakamura, Tokyo (JP)

(73) Assignee: Olympus Corporation, Tokyo (JP)

(21) Appl. No.: 16/297,611

(22) Filed: Mar. 9, 2019

Related U.S. Application Data
(63) Continuation of application No. PCT/JP2016/084388, filed on Nov. 21, 2016.

(51) Int. Cl. A61B 17/3201; B26B 17/00; A61B 17/28

(52) U.S. Cl. CPC A61B 17/3201; A61B 2018/00601; A61B 17/28; B26B 17/00

(57) ABSTRACT

A scissor forceps comprises a base and a pair of blades configured to be pivotally attached to one another via a swing shaft attached to the base in an overlapping relationship. A drive mechanism is used to drive the blades and includes a power transmitting member that transmits a pulling force. A swing mechanism is disposed on a proximal side of the blades relative to the swing shaft converting a portion of the pulling force being transmitted by the power transmitting member to a first force that swings the blades. A pressing mechanism that converts another portion of the pulling force to a second force that separates the blades in the axial direction of the swing shaft on the proximal side of the blades. The swing mechanism includes cam grooves formed through the blades along directions that intersect one another on the proximal side of each of the blades.

### CROSS-REFERENCE TO RELATED APPLICATION

This application is a continuation application of PCT Application No. PCT/JP2016/084388 filed on Nov. 21, 2016, which is hereby incorporated by reference in its entirety.

### TECHNICAL FIELD

The disclosed technology relates to one or more scissor forceps.

### DESCRIPTION OF THE RELATED ART

Conventionally, a scissor forceps are known in which a slope is made at part of a link mechanism for opening and closing a pair of blades that are pivotally coupled and blade surfaces of both blades are biased in such a direction as to be pressed against each other when the pair of blades are closed. For example, refer to PTL 1 or Japanese Patent Laid-open No. 2012-165812

However, in the scissor forceps of PTL 1, a link gets on the slope made in the other link, and thereby a biasing force that presses the blade surfaces of both blades against each other is generated. Therefore, there is inconvenience that the biasing force is not generated and a gap between the blade surfaces is made around the roots of the blades, at which the blade surfaces begin to get contact with each other first, and a cutting target gets stuck between the blade surfaces and becomes impossible to cut.

### BRIEF SUMMARY OF EMBODIMENTS

The disclosed technology is made in view of the circumstances described hereinbefore and intends to provide one or more scissor forceps that can always generate a biasing force that presses blade surfaces of a pair of blades against each other when the blades are closed, and can cut a cutting target more surely.

One aspect of the disclosed technology is directed to a scissor forceps including a base, a pair of blades that are attached relatively pivotally around a swing shaft attached to the base in the state of being disposed to be overlapping in the direction of the swing shaft and are restricted from moving in the direction along the swing shaft at the position of the swing shaft, and a drive mechanism that being used to drive the blades. The drive mechanism includes a power transmitting member that transmits a pulling force, a swing mechanism that is disposed on the proximal side of the blades relative to the swing shaft and converts part of the pulling force transmitted by the power transmitting member to a force that swings the blades, and a pressing mechanism that converts the other part of the pulling force to a force that separates the blades in the overlapping direction on the proximal side of the blades relative to the swing shaft at all swinging positions of the blades based on the swing mechanism.

According to the present aspect, when the pulling force is applied to the power transmitting member configuring the drive mechanism, the swing mechanism is actuated by part of the pulling force transmitted by the power transmitting member and the pair of blades are relatively swung around the swing shaft. Thereby, the blade edges are opened and closed. The other part of the pulling force separates the blades in the overlapping direction on the proximal side relative to the swing shaft. Therefore, with the position of the swing shaft, at which movement in the axial direction is restricted, being a fulcrum, the parts of the blades on the distal side relative to the swing shaft are biased in such a direction as to be brought close to each other at all swinging positions of the blades.

Due to this, the blade edges made in the blades press each other at all swinging positions and thus the cutting target can be cut more surely without getting stuck between the blades.

In the aspect described hereinbefore, the swing mechanism may include cam grooves made to extend along directions that intersect each other on the proximal side of each of the blades relative to the swing shaft and a pin that penetrates the intersection position of the cam grooves and is supported by the blades in the manner of a cantilever. The power transmitting member is connected to the tip side of the cantilever. Furthermore, the pressing mechanism may include a moment transmitting portion that transmits, to one of the blades, a moment generated on the pin due to the pulling force transmitted by the power transmitting member.

By doing this, when the pulling force in a direction intersecting the longitudinal axis of the pin is transmitted, by the power transmitting member, to the pin disposed to penetrate through the two cam grooves extending in directions that mutually intersect in each of the pair of blades, the relative position between the cam grooves is changed and thereby the pair of blades are rotated around the swing shaft, so that the blade edges are opened and closed.

The pin is configured in the manner of the cantilever by being supported by the cam grooves. Thus, a moment is generated in such a direction as to tilt the pin when the pulling force acts on the tip side of the cantilever. This moment is transmitted to one of the blades by the moment transmitting portion and thereby the one of the blades is separated from the other of the blades on the proximal side relative to the swing shaft. Thus, with the position of the swing shaft, at which movement in the axial direction is restricted, being a fulcrum, the parts of the blades on the distal side relative to the swing shaft are biased in such a direction as to be brought close to each other at all swinging positions of the blades.

Furthermore, in the aspect described hereinbefore, the moment transmitting portion may be a large diameter portion that is disposed around the pin in such a manner as to protrude in the radial direction and is disposed between the blades and is brought into tight contact with a surface of one of the blades.

By doing this, when the moment acts in such a direction as to tilt the pin, one of the blades is pressed by the large diameter portion in tight contact with the surface of the one of the blades. This can easily transmit the moment generated on the pin to the blade.

Moreover, in the aspect described hereinbefore, the power transmitting member may be a wire and the moment transmitting portion may be a pulley that is disposed rotatably around the pin and around which the wire is wound.

By doing this, the pulley can be used as a movable pulley and the pulling force can be amplified, so that the pair of blades can be relatively swung with a small pulling force. In addition, the blade edges of the blades can be brought into tight contact with each other.

Furthermore, in the aspect described hereinbefore, the power transmitting member may be a wire, and the moment transmitting portion may be a pulley that is disposed rotatably around the pin and is composed of an elastic material around which the wire is wound, and the moment transmitting portion may be expanded in the axial direction when being contracted in the radial direction by the pulling force.

By doing this, when the pulling force acts on the wire, the pulling force is amplified by the pulley that is a movable pulley. In addition, the pulley is expanded in the axial direction by being contracted in the radial direction and the blade in tight contact with a side surface of the pulley in the axial direction is pressed, so that one of the blades is separated from the other of the blades on the proximal side relative to the swing shaft. Thus, with the position of the swing shaft, at which movement in the axial direction is restricted, being a fulcrum, the parts of the blades on the distal side relative to the swing shaft are biased in such a direction as to be brought close to each other at all swinging positions of the blades.

Moreover, in the aspect described hereinbefore, the swing mechanism may be made by pivotally coupling each of one ends of two coupled links having the other ends coupled mutually pivotally by a coupling shaft to the proximal side of a respective one of the blades relative to the swing shaft and connecting the power transmitting member to the coupling shaft. In addition, the pressing mechanism may be made by configuring the coupling shaft by two small links that have one ends coupled mutually pivotally and each have the other end pivotally coupled to a fitted pin fitted to a hole made at a proximal end of the coupled link and connecting the power transmitting member to a coupling portion between the small links, and the coupling portion may be disposed on the distal side relative to the fitted pins.

By doing this, by causing the coupling shaft that couples the coupled links to function as a toggle mechanism by the two small links and pulling the coupling portion of the small links by the pulling force, the interval between the coupled links is increased on the proximal side relative to the swing shaft and thereby the pair of blades coupled to the coupled links are separated on the proximal side relative to the swing shaft. Thus, with the position of the swing shaft being a fulcrum, the parts of the blades on the distal side relative to the swing shaft are biased in such a direction as to be brought close to each other at all swinging positions of the blades.

Furthermore, in the aspect described hereinbefore, the swing mechanism may be made by pivotally coupling each of one ends of two coupled links having the other ends coupled mutually pivotally by a coupling shaft to the proximal side of a respective one of the blades relative to the swing shaft and connecting the power transmitting member to the coupling shaft. In addition, the pressing mechanism may configure the coupling shaft bendably and include a moment transmitting portion that transmits, to one of the blades, a moment generated on the coupling shaft due to bending attributed to the pulling force transmitted by the power transmitting member.

By doing so, when the pulling force is applied to the power transmitting member connected to the coupling shaft that couples the coupled links, a moment that bends the coupling shaft is generated and the generated moment is transmitted to one of the blades by the moment transmitting portion. Thereby, the pair of blades are separated on the proximal side relative to the swing shaft and, with the position of the swing shaft being a fulcrum, the parts of the blades on the distal side relative to the swing shaft are biased in such a direction as to be brought close to each other at all swinging positions of the blades.

Moreover, in the aspect described hereinbefore, at least one of the blades may have an energy emitting portion.

By doing this, at the time of cutting the cutting target by closing the blades, the cutting target can be effectively cut through emission of energy by the energy emitting portion.

The disclosed technology provides an effect that a biasing force that presses blade surfaces of a pair of blades against each other can be always generated when the blades are closed and a cutting target can be cut more surely.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a front view of a distal portion depicting the state in which the scissor forceps according to one embodiment of the disclosed technology are opened.

FIG. 2 is a partial longitudinal sectional view of a distal portion of the scissor forceps of FIG. 1.

FIG. 3 is a partial enlarged longitudinal sectional view of the distal portion of the scissor forceps of FIG. 2.

FIG. 4 is a front view of the distal portion depicting the state in which the scissor forceps of FIG. 1 are closed.

FIG. 5 is a partial longitudinal sectional view of the distal portion of the scissor forceps of FIG. 4.

FIG. 6 is a front view of a distal portion depicting a first modification example of the scissor forceps of FIG. 1.

FIG. 7 is a side view of the distal portion of the scissor forceps of FIG. 6.

FIG. 8 is a side view depicting the state in which a pulling force is made to act on the scissor forceps of FIG. 6.

FIG. 9 is a side view of a distal portion depicting a second modification example of the scissor forceps of FIG. 1.

FIG. 10 is a partial enlarged view of the distal portion of the scissor forceps of FIG. 9.

FIG. 11 is a side view of a distal portion depicting a third modification example of the scissor forceps of FIG. 1.

FIG. 12 is a front view of the distal portion of the scissor forceps of FIG. 11.

FIG. 13 is a side view of a distal portion depicting a fourth modification example of the scissor forceps of FIG. 1.

FIG. 14 is an enlarged view depicting the state in which a pulling force is made to act on the scissor forceps of FIG. 13.

FIG. 15 is a perspective view of a distal portion depicting a fifth modification example of the scissor forceps of FIG. 1.

FIG. 16 is a cross-sectional view of blades of the scissor forceps of FIG. 15.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

A scissor forceps 1 according to one embodiment of the disclosed technology will be described below with reference to the drawings.

The scissor forceps 1 according to the present embodiment include a pair of blades 2a and 2b disposed at the distal end of an insertion portion inserted in a channel of an endoscope and a drive mechanism 3 that being used to drive these blades 2a and 2b, for example.

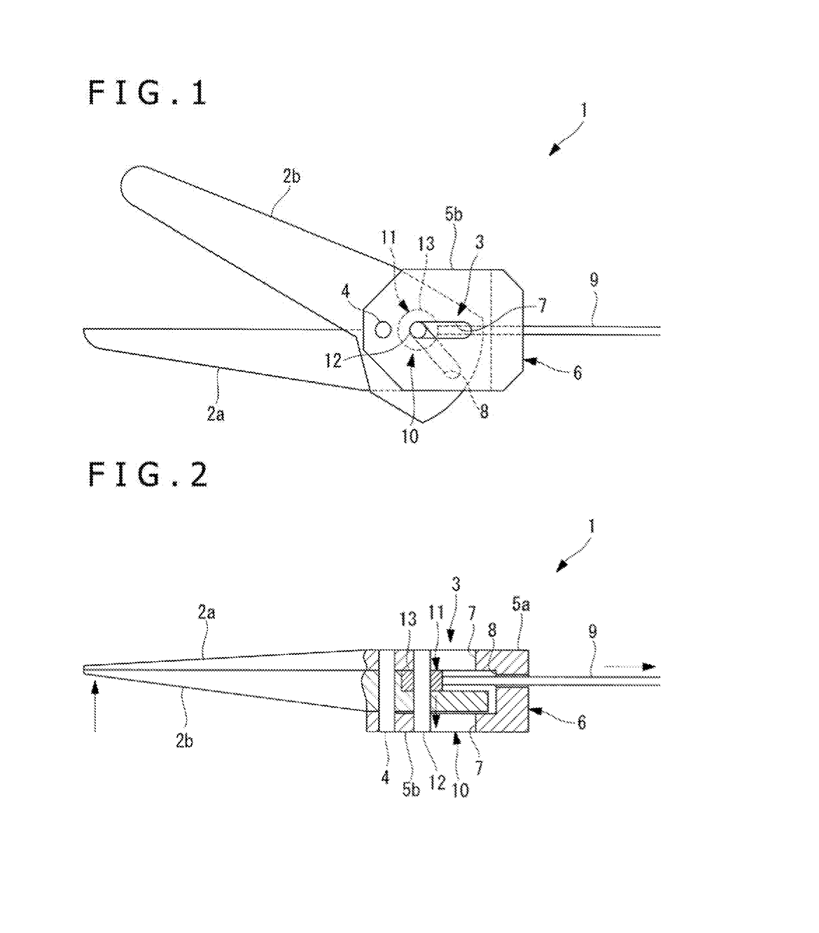

The blades 2a and 2b are each formed into a flat plate shape and, in the state of being overlapped with each other in the plate thickness direction, are mutually pivotally coupled by a swing shaft 4 that penetrates in the plate thickness direction at a halfway position in the length direction. These blades 2a and 2b are relatively swung between the state of being opened as depicted in FIG. 1 and the state of being closed as depicted in FIG. 4.

The respective blades 2a and 2b have blade edges along rims that mutually overlap in the closed state on the distal side relative to the swing shaft 4. By disposing tissue that is a cutting target between the blades 2a and 2b in the opened state and making a transition to the closed state, the blade edges are made to sequentially intersect from a position close to the swing shaft 4 and the tissue is cut.

On the proximal side of one blade (hereinafter, referred to also as first blade) 2a relative to the swing shaft 4, as depicted in FIG. 2, two flat plate portions 5a and 5b are disposed with the intermediary of an interval in the plate thickness direction and are coupled on the proximal side, and thereby a base portion 6, or base, formed into a longitudinal sectional shape of a substantially U-shape is disposed. The base portion 6 is attached to the distal end of the insertion portion.

The proximal side of the other blade (hereinafter, referred to also as second blade) 2b is housed between the two flat plate portions 5a and 5b of the first blade 2a. In the two flat plate portions 5a and 5b of the first blade 2a, first long holes 7, or cam grooves, that penetrate the respective flat plate portions 5a and 5b in the plate thickness direction and extend along a straight line including the swing shaft 4 to the proximal side relative to the swing shaft 4 are formed. Furthermore, in the second blade 2b, a second long hole 8, or cam groove, that extends in a direction intersecting the first long holes 7 is formed to penetrate in the plate thickness direction.

For the two blades 2a and 2b, at the position of the swing shaft 4, relative movement in the plate thickness direction is constrained and only relative swinging around the swing shaft 4 is permitted.

Meanwhile, between the base portion 6 of the first blade 2a and the second blade 2b, a gap is formed in the plate thickness direction on the proximal side relative to the swing shaft 4. This allows the second blade 2b to be displaced in such a direction as to decrease the gap when receiving a force in the plate thickness direction on the proximal side relative to the swing shaft 4.

The drive mechanism 3 is disposed at the proximal end of the insertion portion and includes a power transmitting member 9 formed of an elongated member such as a wire that transmits a pulling force from a drive portion that is not depicted in the diagram, such as a handle that generates the pulling force, to the distal end of the insertion portion, a swing mechanism 10 that swings the second blade 2b around the swing shaft 4 relative to the first blade 2a by part of the pulling force transmitted by the power transmitting member 9, and a pressing mechanism 11 that biases the first blade 2a and the second blade 2b in such a direction as to press the blade edges of the first blade 2a and the second blade 2b against each other in the plate thickness direction by the other part of the pulling force.

The swing mechanism 10 includes the first long holes 7 and the second long hole 8 formed in the two blades 2a and 2b and a pin 12 disposed to penetrate the intersection position of these first long holes 7 and the second long hole 8 in the plate thickness direction.

Furthermore, the pressing mechanism 11 is configured by a large diameter portion 13, or moment transmitting portion, with an outer flange shape that is disposed at a position sandwiched by the two blades 2a and 2b and protrudes in the radial direction.

The distal end of the power transmitting member 9 is attached to the large diameter portion 13 and the pulling force transmitted by the power transmitting member 9 acts on the large diameter portion 13.

The operation of the scissor forceps 1 according to the present embodiment configured in this manner will be described below.

To cut tissue by using the scissor forceps 1 according to the present embodiment, the scissor forceps 1 in which the two blades 2a and 2b are set to the closed state are inserted into a body from the distal end through a channel of an endoscope disposed in the body, and the two blades 2a and 2b are made opposed to an affected site in the body. In this state, the drive portion at the proximal end of the insertion portion disposed outside the body is actuated and a pressing force is transmitted to the large diameter portion 13 disposed between the blades 2a and 2b by the power transmitting member 9.

Thereby, the pin 12 around which the large diameter portion 13 is disposed is moved by the pressing force toward the distal side along the first long holes 7 made in the first blade 2a and the second long hole 8 made in the second blade 2b is moved by the pin 12. As a result, the second blade 2b is swung around the swing shaft 4 relative to the first blade 2a and the first blade 2a and the second blade 2b are opened as depicted in FIG. 1.

Then, while the tissue is checked by the endoscope, the tissue that is the cutting target is disposed between the first blade 2a and the second blade 2b that are opened and a pulling force depicted by an arrow in FIG. 2 is generated by the drive portion. Thereby, the generated pulling force is transmitted to the large diameter portion 13 by the power transmitting member 9.

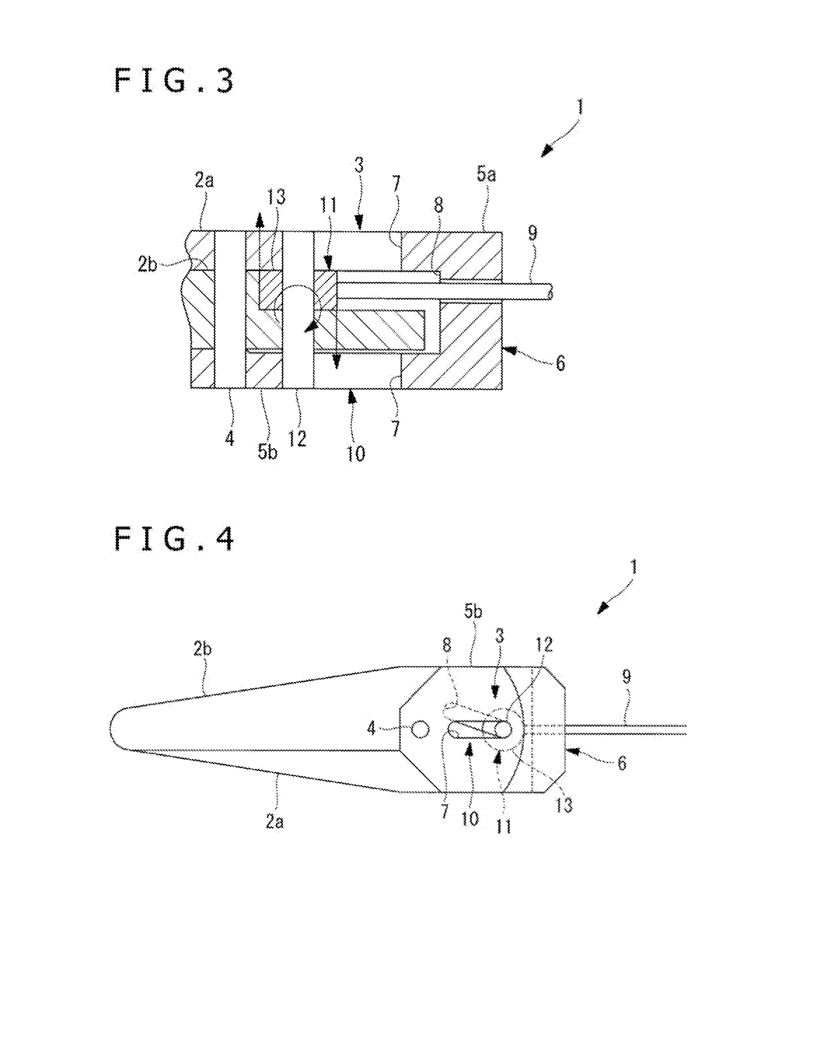

In this case, according to the scissor forceps 1 in accordance with the present embodiment, as depicted in FIG. 3, on one side of the pin 12 in the longitudinal direction, the pin 12 is sandwiched by the inner wall of the first long hole 7 and the inner wall of the second long hole 8 to be supported due to being disposed at the intersection position of the first long hole 7 and the second long hole 8 that mutually intersect. Meanwhile, on the other side of the pin 12 in the longitudinal direction, the pin 12 is disposed only in the first long hole 7 and is not supported in the direction of the pulling force. As a result, the pin 12 is supported in a cantilevered manner. Due to action of the pulling force orthogonal to the pin 12 on the large diameter portion 13 disposed on the free end side of the cantilever, a moment is generated on the pin 12 in such a direction as to tilt the pin 12 as depicted by an arrow in FIG. 3.

Then, the generated moment is received by pressing shoulder portions of the large diameter portion 13 against the first blade 2a and the second blade 2b. Thus, from the shoulder portions of the large diameter portion 13, a pressing force that separates the first blade 2a and the second blade 2b in the plate thickness direction acts in directions depicted by arrows in FIG. 3. This pressing force always acts in the state in which a pulling force acts on the large diameter portion 13. Therefore, the pressing force acts whichever position along the first long hole 7 the pin 12 is disposed at, i.e. whatever relative angle the first blade 2a and the second blade 2b are disposed at.

As described hereinbefore, in the scissor forceps 1 according to the present embodiment, the position of the first blade 2a and the second blade 2b in the longitudinal direction of the swing shaft 4 is constrained at the position of the swing shaft 4 and a gap is made on the proximal side relative to the swing shaft 4. Therefore, when a pressing force that separates the first blade 2a and the second blade 2b acts on the proximal side of the swing shaft 4, a biasing force that presses the first blade 2a and the second blade 2b on the distal side relative to the swing shaft 4 acts, with the position of the swing shaft 4 being a fulcrum.

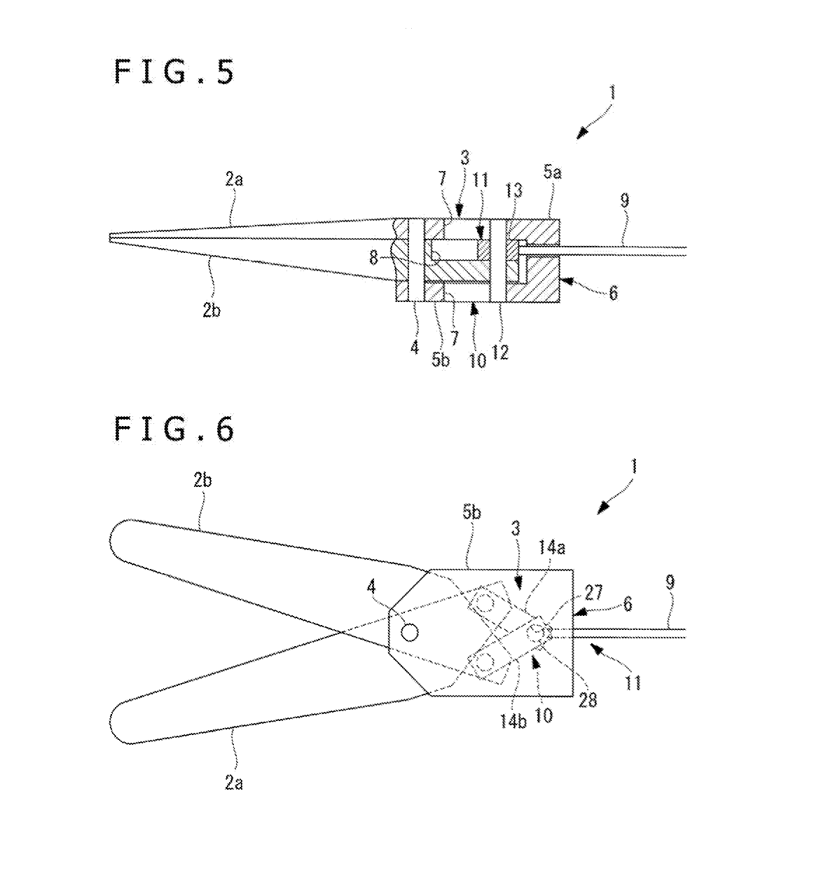

Then, by a pulling force applied to the large diameter portion 13, the pin 12 around which the large diameter portion 13 is disposed is moved toward the proximal side along the first long holes 7 as depicted in FIG. 4 and FIG. 5. Due to this, the second long hole 8 intersecting the first long holes 7 is moved in such a manner as to follow the pin 12, and the second blade 2b, in which the second long hole 8 is made, is swung in the closing direction around the swing shaft 4 relative to the first blade 2a.

As described hereinbefore, according to the scissor forceps 1 in accordance with the present embodiment, when a pulling force is transmitted by the power transmitting member 9, by the transmitted pulling force, the first blade 2a and the second blade 2b are relatively swung in the closing direction around the swing shaft 4 while the first blade 2a and the second blade 2b on the distal side relative to the swing shaft 4 are mutually pressed in the plate thickness direction at all swinging positions. Due to this, the first blade 2a and the second blade 2b are closed while the blade edges of the first blade 2a and the second blade 2b disposed on the distal side relative to the swing shaft 4 are mutually pressed. Thus, there is an advantage that tissue disposed between the first blade 2a and the second blade 2b can be cut more surely.

In the present embodiment, the swing mechanism 10 is configured by the first long holes 7 and the second long hole 8 that mutually intersect and the pin 12 that penetrates the intersection position of them. However, instead of this, the swing mechanism 10 may be configured by links 14a and 14b, or coupled links, as depicted in FIG. 6 to FIG. 8.

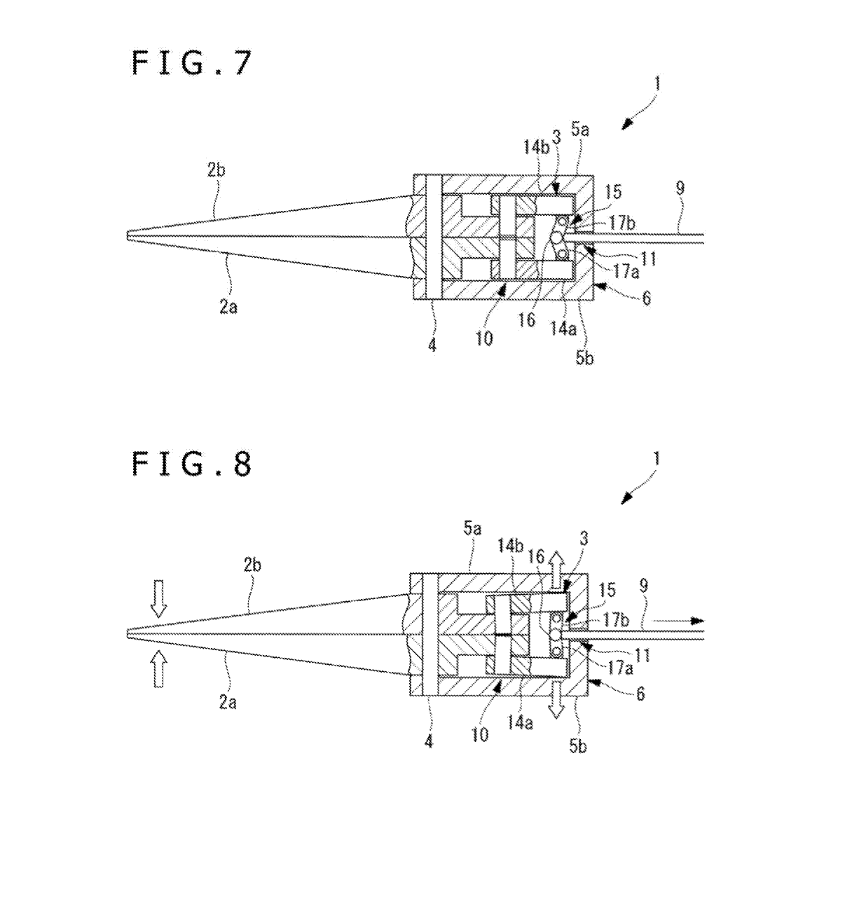

In the example depicted in FIG. 6 and FIG. 7, a four-link structure may be configured by pivotally coupling the respective one ends of the two bar-shaped links 14a and 14b, the two bar-shaped links 14a and 14b having the other ends pivotally coupled by a coupling shaft 15, to the proximal ends of the first blade 2a and the second blade 2b, and the first blade 2a and the second blade 2b may be swung in the closing direction through the links 14a and 14b by applying a pulling force to the power transmitting member 9 attached to the coupling shaft 15.

In this case, as depicted in FIG. 7, the coupling shaft 15 formed of two small links 17a and 17b or an elastically deformable shaft disposed in the state in which a joint portion 16, or coupling portion, at the center is always bent toward the distal side may be employed as the pressing mechanism 11 and the pulling force transmitted by the power transmitting member 9 may be applied to the joint portion 16. If the coupling shaft 15 is formed of the small links 17a and 17b, one ends of the small links 17a and 17b are coupled mutually pivotally to configure the joint portion 16 and the respective other ends are pivotally coupled to holes 27 made at the proximal ends of the links 14a and 14b by fitted pins 28.

Due to this, when a pulling force acts on the joint portion 16, a so-called toggle mechanism in which the joint portion 16 is unbent and the coupling shaft 15 stretches is configured and the two links 14a and 14b are separated on the proximal side relative to the swing shaft 4 as depicted in FIG. 8. As a result, the first blade 2a and the second blade 2b on the distal side relative to the swing shaft 4 can be mutually pressed in the plate thickness direction.

That is, also by such a configuration, part of the pulling force transmitted by the power transmitting member 9 always generates a force that swings the first blade 2a and the second blade 2b in the closing direction and a biasing force that causes the first blade 2a and the second blade 2b to be mutually pressed in the plate thickness direction, and a high shear force is generated on tissue between the first blade 2a and the second blade 2b, so that the tissue can be cut more surely.

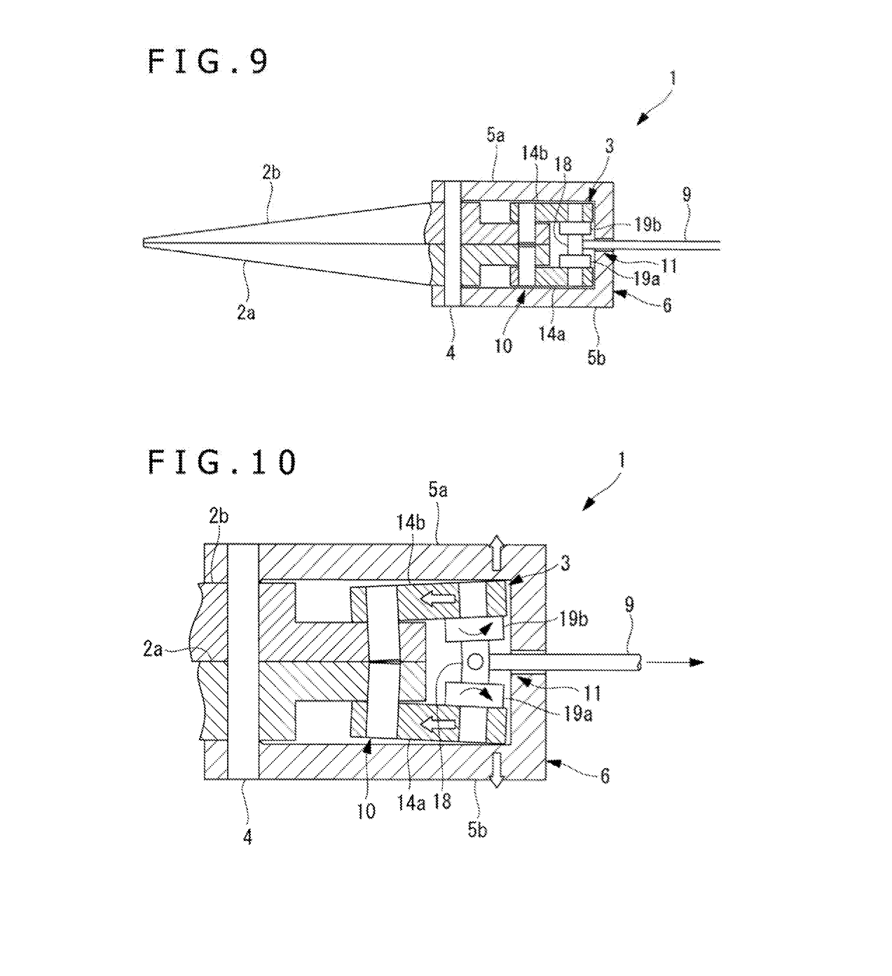

Furthermore, instead of what is obtained by coupling the two small links 17a and 17b having the joint portion 16 as the coupling shaft 15, as depicted in FIG. 9 and FIG. 10, a coupling shaft 18 that can be deformed by a pulling force may be employed and large diameter portions 19a and 19b, or moment transmitting portions, that protrude in the radial direction and have an outer flange shape may be disposed around this coupling shaft 18. As depicted in FIG. 10, when the coupling shaft 18 is deformed by a pulling force, the large diameter portions 19a and 19b rotate and a force in such a direction as to separate the first blade 2a and the second blade 2b is generated from shoulder portions of the large diameter portions 19a and 19b.

As a result, also by such a configuration, part of the pulling force transmitted by the power transmitting member 9 always generates a force that swings the first blade 2a and the second blade 2b in the closing direction and a biasing force that causes the first blade 2a and the second blade 2b to be mutually pressed in the plate thickness direction, and a high shear force is generated on tissue between the first blade 2a and the second blade 2b, so that the tissue can be cut more surely.

In this case, the coupling shaft 18 may be what is obtained by coupling two small links 17a and 17b by the joint portion 16 or a monolithic coupling shaft composed of an elastically deformable material may be employed.

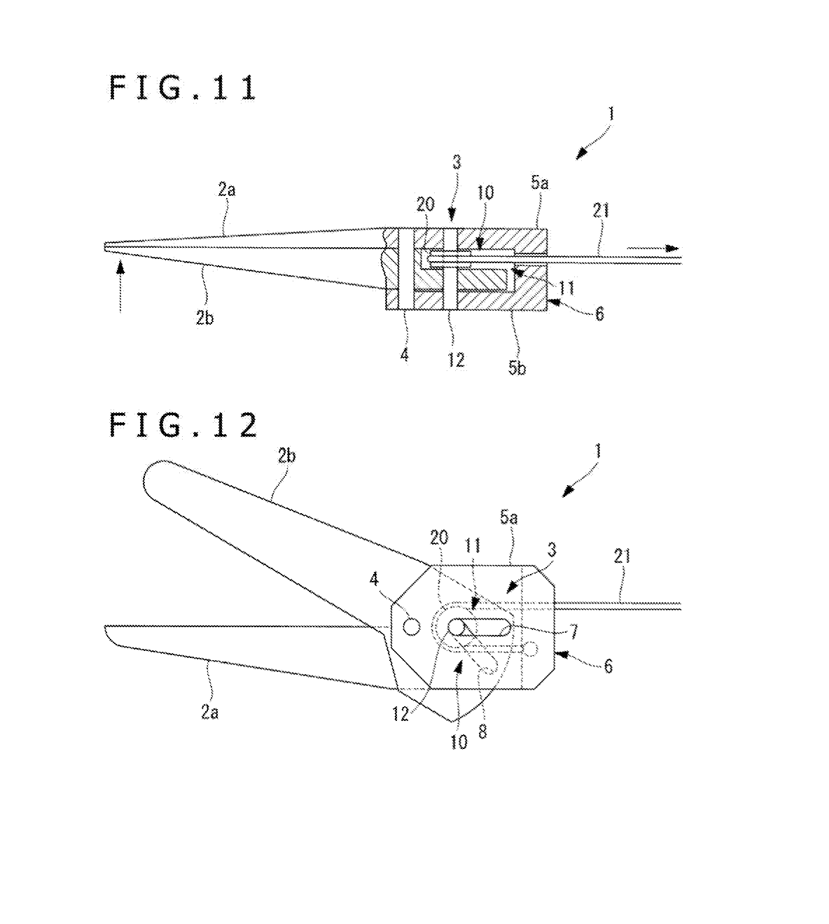

Moreover, as depicted in FIG. 11 and FIG. 12, a pulley 20, or moment transmitting portion, attached to the pin 12 may be employed as the large diameter portion 13 in FIG. 1 and a wire 21 that is wound around the pulley 20 and has a distal end attached to the first blade 2a may be employed as the power transmitting member.

By doing this, the pulley 20 that moves together with the pin 12 can be caused to function as a movable pulley and a pulling force applied to the wire 21 can be amplified to be applied to the pin 12. Due to this, there is an advantage that the pulling force applied to the proximal end of the wire 21 can be reduced and cutting work can be easily carried out with a small pulling force.

In this case, the amplification rate of the pulling force may be increased by disposing an attached pulley (diagrammatic representation is omitted) attached to the first blade 2a or the second blade 2b rotatably around an axis line parallel to the pin 12 and winding plural wires 21 between the pulley 20 attached to the pin 12 and the attached pulley.

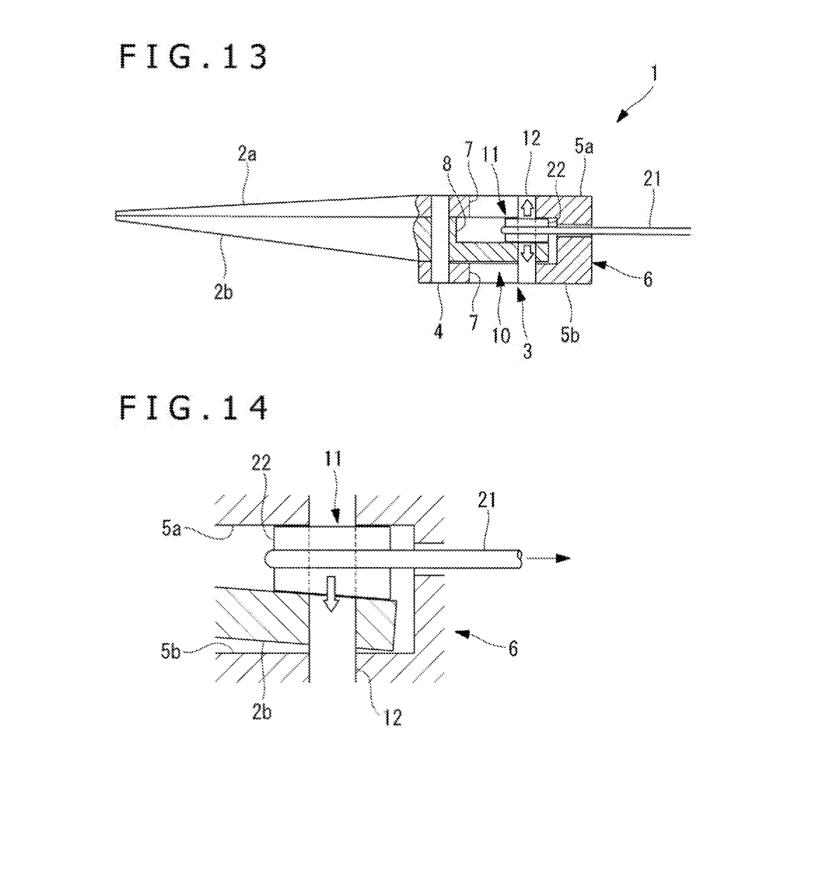

Furthermore, instead of the pulley 20 in FIG. 11, as depicted in FIG. 13 and FIG. 14, a pulley 22 composed of an elastic material that contracts in the radial direction and expands in the axial direction when a pulling force is applied to the wire 21 may be employed.

The first blade 2a and the second blade 2b can be pressed in the separating direction by the expanding pulley 22 and the first blade 2a and the second blade 2b on the distal side relative to the swing shaft 4 can be mutually pressed in the plate thickness direction.

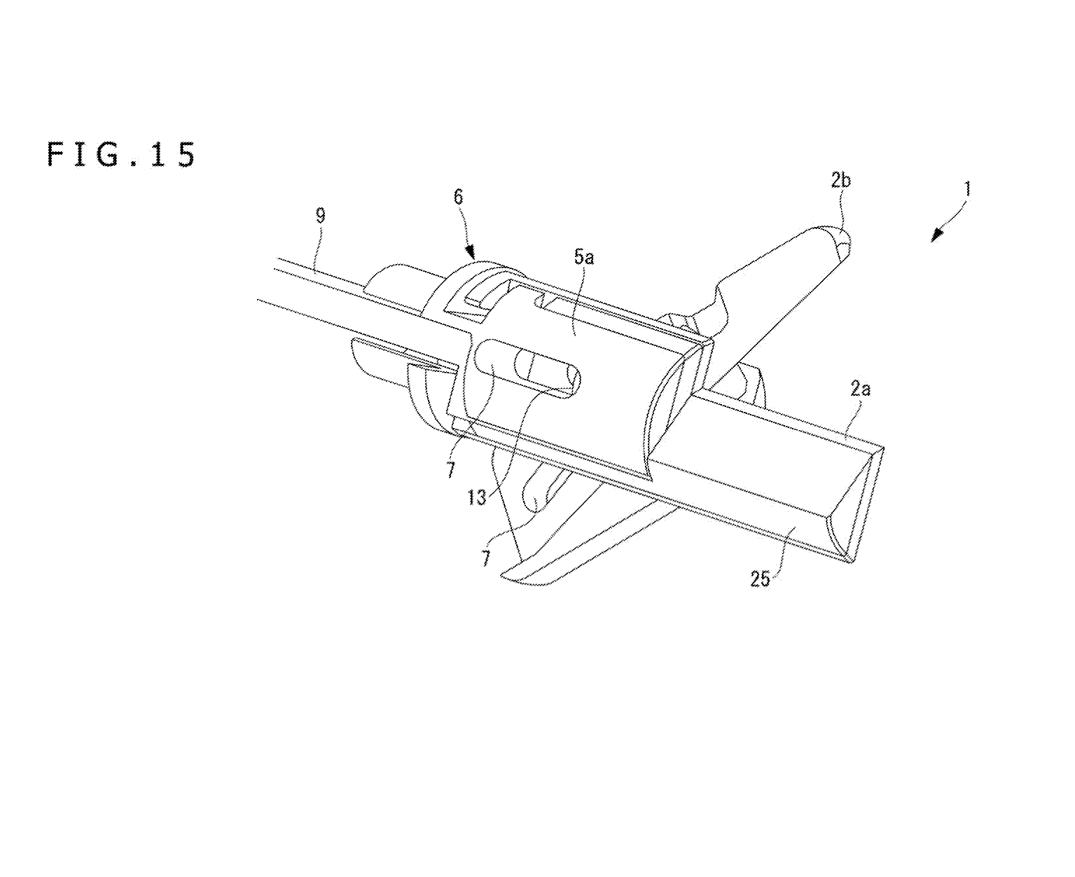

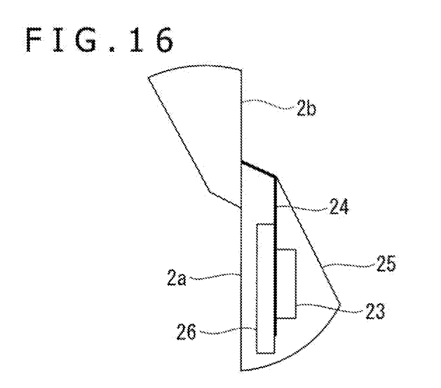

Moreover, as depicted in FIG. 15 and FIG. 16, an energy emitting portion 23 that applies some kind of energy such as Joule heat, high-frequency wave, and vibration to blade edges may be mounted on the distal side of the first blade 2a attached to the distal end of the insertion portion relative to the swing shaft 4. In the example depicted in FIG. 15 and FIG. 16, a thin plate 24 that is stuck to the vicinity of the blade edge and is composed of a material with high energy conductivity, such as copper, is stuck to the surface of the first blade 2a, and the energy from the energy emitting portion 23 stacked on the first blade 2a can be supplied to the blade edge in a concentrated manner via the thin plate 24. In FIG. 16, numeral 26 denotes a component that blocks the energy from the energy emitting portion 23.

Furthermore, by employing what has a cross-sectional shape whose thickness gradually increases from the blade edge side as depicted in FIG. 16 as a cover 25 that covers the energy emitting portion 23, tissue can be cut with a smaller amount of force due to a wedge effect that pushes apart the cut tissue.

An embodiment of a scissor forceps comprises an elongated first blade having a U-shaped first end at a proximal and a free second end opposed from the U-shaped first end. The U-shaped first end is defined by a base. A second blade is pivotally attached to the U-shaped first end of the first blade via a swing shaft and is sandwiched inside the U-shaped first end in an axial direction of the swing shaft. A power transmitting member is configured to transmit a pulling force. A swing mechanism is configured to convert the pulling force to a first force that swings the first and the second blades. The swing mechanism includes a first cam groove formed in the U-shaped first end of the first blade on a proximal side of the first blade relative to the swing shaft. A second cam groove is formed on a proximal side of the second blade relative to the swing shaft. The second cam groove intersecting to the first cam groove and a pin penetrates the first and the second blades and is configured to move along the first and second cam grooves. The pin is configured to be supported by the first and second blades so as to define a cantilever. A pressing mechanism is configured to convert the pulling force to a second force that separates the first and second blades in the axial direction of the swing shaft on the proximal side of the first and second blades. The pressing mechanism includes a moment transmitting portion formed around a tip side of the pin and connected to the power transmitting member. The moment transmitting portion is configured to transmit a moment generated on the pin by the pulling force to one of the first and second blades.

The moment transmitting portion is a large diameter portion that is disposed around the pin in such a manner as to protrude in a radial direction and is disposed between the first and second blades and is brought into tight contact with a surface of one of the blades. The power transmitting member is defined by a wire. The moment transmitting portion is defined by a pulley that is disposed rotatably around the pin and around which the wire is wound. The power transmitting member is defined by a wire and the moment transmitting portion is defined by an elastic pulley that is disposed rotatably around the pin. The wire is wound around the elastic pulley and the moment transmitting portion expands in the axial direction when being contracted in a radial direction by the pulling force. At least one of the first and second blades has an energy emitting portion.

Another embodiment of a scissor forceps comprises a first blade having a first plate portion and a second plate portion. The first plate portion and the second plate portion respectively having proximal ends. The first plate portion and the second plate portion are connected at the both proximal ends via a base. A second blade relatively is pivotally attached to the first blade around a swing shaft and is sandwiched between the first and the second plate portion in an axial direction of a swing shaft. A power transmitting member is configured to transmit a pulling force. A swing mechanism is configured to convert the pulling force to a first force that swings the first and the second blades. The swing mechanism includes two coupled links respectively having proximal ends and distal ends. The proximal ends of the two coupled links pivotally coupled at each other by a coupling shaft. The coupling shaft is connected to the power transmitting member. the distal ends of the two coupled links are disposed a proximal side of the first and second blades relative to the swing shaft. The distal ends of the two coupled links are pivotally coupled by a respective one of the first and second blades. A pressing mechanism is configured to convert the pulling force to a second force that separates the first and second blades in the axial direction of the swing shaft on the proximal side of the first and second blades. The pressing mechanism is defined by the coupling shaft. The coupling shaft includes two second links respectively having first ends and second ends. The first ends of the second links are pivotally coupled to each other and are connected to the power transmitting member. The second ends of the second links are pivotally fitted to holes formed at the proximal ends of the coupled link. The at least one of the first and second blades has an energy emitting portion.

A further embodiment of a scissor forceps comprises a first blade having a first plate portion and a second plate portion. The first plate portion and the second plate portion respectively having proximal ends. The first plate portion and the second plate portion are connected at the both proximal ends via a base. A second blade relatively is pivotally attached to the first blade around a swing shaft and is sandwiched between the first and the second plate portion in an axial direction of a swing shaft. A power transmitting member is configured to transmit a pulling force. A swing mechanism is configured to convert the pulling force to a first force that swings the first and the second blades. The swing mechanism includes two coupled links respectively having proximal ends and distal ends. The proximal ends of the two coupled links pivotally coupled at each other by a coupling shaft. The coupling shaft is connected to the power transmitting member. The distal ends of the two coupled links are disposed a proximal side of the first and second blades relative to the swing shaft. The distal ends of the two coupled links are pivotally coupled by a respective one of the first and second blades. A pressing mechanism is configured to convert the pulling force to a second force that separates the first and second blades in the axial direction of the swing shaft on the proximal side of the first and second blades. The pressing mechanism is defined by the coupling shaft and a moment transmitting portion. The coupling shaft is bendable. The moment transmitting portion is configured to transmit a moment to one of the first and second blades. The moment is generated on the coupling shaft by bending the coupling shaft based on the pulling force. The at least one of the blades has an energy emitting portion.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one”, “one or more” or the like; and adjectives such as “conventional”, “traditional”, “normal”, “standard”, “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more”, “at least”, “but not limited to” or other like phrases in some instances shall not be read to mean that the narrower case is intended or required in instances where such broadening phrases may be absent. Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

### NUMERAL REFERENCE LIST

1 Scissor forceps
2a First blade (Blade)
2b Second blade (Blade)
3 Drive mechanism
4 Swing shaft
6 Base portion (Base)
7 First long hole (Cam groove)
8 Second long hole (Cam groove)
9 Power transmitting member
10 Swing mechanism
11 Pressing mechanism
12 Pin
13, 19a, 19b Large diameter portion (Moment transmitting portion)
14a, 14b Link (Coupled link)
15, 18 Coupling shaft
16 Joint portion (coupling portion)
17a, 17b Small link
20, 22 Pulley (Moment transmitting portion)
21 Wire (Power transmitting member)
23 Energy emitting portion
27 Hole
28 Fitted pin

What is claimed is:

1. A scissor forceps comprising:
a base;
a pair of blades configured to be pivotally attached to one another via a swing shaft attached to the base in an overlapping relationship in an axial direction of the swing shaft; and
a drive mechanism being used to drive the blades wherein
the drive mechanism includes
a power transmitting member that transmits a pulling force,
a swing mechanism disposed on a proximal side of the blades relative to the swing shaft converting a portion of the pulling force being transmitted by the power transmitting member to a first force that swings the blades, and
a pressing mechanism that converts another portion of the pulling force to a second force that separates the blades in the axial direction of the swing shaft on the proximal side of the blades,
the swing mechanism includes
cam grooves formed through the blades along directions that intersect one another on the proximal side of each of the blades relative to the swing shaft, and
a pin that penetrates an intersection position of the cam grooves and being supported by the blades so as to define a cantilever, the power transmitting member being connected to a tip side of the cantilever, and
the pressing mechanism includes a moment transmitting portion that transmits, to one of the blades, a moment generated on the pin as a result of the pulling force transmitted by the power transmitting member.

2. The scissor forceps of claim 1, wherein
the moment transmitting portion is a large diameter portion that is disposed around the pin in such a manner as to protrude in a radial direction and is disposed between the blades and is brought into tight contact with a surface of one of the blades.

3. The scissor forceps of claim 2, wherein
the power transmitting member is defined by a wire, and
the moment transmitting portion is defined by a pulley that is disposed rotatably around the pin and around which the wire is wound.

4. The scissor forceps of claim 2, wherein
the power transmitting member is defined by a wire, and
the moment transmitting portion is defined by a pulley that is disposed rotatably around the pin and is made of an elastic material, the wire being wound around the elastic pulley, and the moment transmitting portion expands in the axial direction when being contracted in a radial direction by the pulling force.

5. The scissor forceps of claim 1, wherein
at least one of the blades has an energy emitting portion.

6. A scissor forceps comprising:
a base;
a pair of blades configured to be pivotally attached to one another via a swing shaft attached to the base in an overlapping relationship in an axial direction of the swing shaft; and
a drive mechanism being used to drive the blades wherein
the drive mechanism includes
a power transmitting member that transmits a pulling force,
a swing mechanism disposed on a proximal side of the blades relative to the swing shaft converting a portion of the pulling force being transmitted by the power transmitting member to a first force that swings the blades, and
a pressing mechanism that converts another portion of the pulling force to a second force that separates the blades in the axial direction of the swing shaft on the proximal side of the blades,
the swing mechanism includes two coupled links that is pivotally coupled at each of respective one ends and having the other ends being pivotally coupled by a coupling shaft to the proximal side of a respective one of the blades relative to the swing shaft and connecting the power transmitting member to the coupling shaft,
the pressing mechanism is defined by the coupling shaft having two small links which have one ends coupled pivotally and each have the other end pivotally coupled to a second pin fitted to a hole made at a proximal end of the coupled link and connecting the power transmitting member to a coupling portion between the small links wherein the coupling portion is located on a distal side of the second pins.

7. The scissor forceps of claim 6, wherein
at least one of the blades has an energy emitting portion.

8. A scissor forceps comprising:
a base;
a pair of blades configured to be pivotally attached to one another via a swing shaft attached to the base in an overlapping relationship in an axial direction of the swing shaft; and
a drive mechanism being used to drive the blades wherein
the drive mechanism includes
a power transmitting member that transmits a pulling force,
a swing mechanism disposed on a proximal side of the blades relative to the swing shaft converting a portion of the pulling force being transmitted by the power transmitting member to a first force that swings the blades, and
a pressing mechanism that converts another portion of the pulling force to a second force that separates the blades in the axial direction of the swing shaft on the proximal side of the blades,
the swing mechanism includes two coupled links that is pivotally coupled at each of respective one ends and having the other ends being pivotally coupled by a coupling shaft to the proximal side of a respective one of the blades relative to the swing shaft and connecting the power transmitting member to the coupling shaft, and
the pressing mechanism is defined by the coupling shaft bendably and includes a moment transmitting portion that transmits, to one of the blades, a moment generated on the coupling shaft as a result of bending attributed to the pulling force transmitted by the power transmitting member.

9. The scissor forceps of claim 8, wherein
at least one of the blades has an energy emitting portion.
